(19) United States
(12) Patent Application Publication
(10) Pub. No.: US 2009/0221693 A1
(43) Pub. Date: Sep. 3, 2009

(54) NOVEL USE OF ORGANIC COMPOUNDS

(76) Inventors: Daniel D'Orazio, Halten (CH); Daniel Raederstorff, Flaxlanden (FR); Goede Schueler, Eimeldingen (DE); Ying Wang-Schmidt, Stallikon (CH); Swen Wolfram, Waldshut-Tiengen (DE)

Correspondence Address: NIXON & VANDERHYE, PC, 901 NORTH GLEBE ROAD, 11TH FLOOR, ARLINGTON, VA, 22203, US

(21) Appl. No.: 12/299,644

(22) PCT Filed: May 2, 2007
(86) PCT No.: PCT/EP2007/003838

§ 371 Date: May 11, 2009

(30) Foreign Application Priority Data
May 10, 2006 (EP) 06009631.0

(51) Int. Cl. A61K 31/352; C07D 311/82; A61P 21/00

(52) U.S. Cl. 514/455; 549/392

(57) ABSTRACT

The present invention refers to compounds of the general formulae I as defined above, preferably α-mangostin, for use as/in a composition (especially foods and dietary supplements, cosmetic, as well as pharmaceutical compositions) for the prevention and improvement of muscular disorders and for the improvement of muscle function and endurance. Other fields of use are eating disorders such as bulimia and anorexia nervosa, use for accelerating wound healing, and/or use for the treatment/prevention of bone disorders such as osteoporosis and osteopenia. The present invention is also directed to dietary compositions such as (fortified) food, beverages, (fortified) feed, food additives, beverage additives, feed additives, clinical nutrition, dietary supplements, functional food, functional feed and nutraceuticals and to pharmaceutical compositions containing such compounds, to methods of preventing and/or treating the above mentioned disorders/diseases, as well as to methods of maintaining the healthy state and/or improving the baseline conditions in mammals including humans and to the compounds of the formula I themselves. Another object of the present invention is the use of such compounds for the manufacture of a composition for the prevention and/or treatment of such disorders/diseases as mentioned above.

The present invention relates to the novel use of organic compounds in the field of (human) nutrition.

### BACKGROUND OF THE INVENTION

Many publications are known, specifically US 2004 146 592. US 2004 146 592 discloses nutraceutical compositions comprising xanthone compounds from processed pericarp from fruit of Garcinia mangostana tree, and at least one juice other than mangosteen juice. Such composition is used for treating cystitis, diarrhea, dysentery, eczema, fever, intestinal disorders, itch, skin disorders, chronic back and neck pain, nausea, chronic vertigo, chronic obstructive pulmonary disease, muscle aches, fatigue, dysthymia, irritable bowel syndrome, hyperlipidemia, insomnia, hypokelemia, degenerative arthritis, Clostridium difficile colitis, numbness of fingers and toes, allergic reactions, malaise, hepatitis, glomerulonephritis, diabetes and dermal rash. The composition has antioxidative, antibacterial, antidepressant, antitubercular, antiviral, anti-inflammatory, cardiotonic, antileukemic, antitumor, antiulcer, antihepatotoxic, antiallergenic and antirhinoviral action.

### SUMMARY OF THE INVENTION

The present invention refers to compounds of the general formulae I as defined below for use as medicament, especially for the treatment of muscular disorders and for the improvement of muscle function. Other fields of use are eating disorders such as bulimia and anorexia nervosa, and bone disorders such as osteoporosis and osteopenia. The compounds may also be used for accelerating wound healing. Preferably the compounds of the general formulae I as defined below are used for (the manufacture of a composition for) the treatment of muscular disorders and for the improvement of muscle function. The most preferred compound of general formula I is α-mangostin.

Thus, especially the supplementation with alpha-mangostin enhances muscle function and endurance and improves the body shape as well as the muscle:fat ratio in mammals.

The present invention is also directed to dietary compositions such as (fortified) food, beverages, (fortified) feed, food additives, beverage additives, feed additives, food premixes, feed premixes, clinical nutrition, dietary supplements, functional food, functional feed and nutraceuticals and to pharmaceutical and body care compositions containing such compounds (especially α-mangostin), to methods for treating muscular disorders (preferred), to methods for the improvement of muscle function (preferred), to methods for treating other conditions such as eating disorders such as bulimia and anorexia nervosa, to methods for accelerating wound healing, and to methods for treating bone disorders such as osteoporosis and osteopenia in mammals including humans and to the compounds of the formula I themselves.

Another object of the present invention is the use of such compounds (especially the use of α-mangostin) for the manufacture of a composition for the treatment of muscular disorders and for the improvement of muscle function, for the treatment of eating disorders such as bulimia and anorexia nervosa, for the acceleration of wound healing, as well as for the treatment of bone disorders such as osteoporosis and osteopenia. Especially preferred is the use of such compounds (especially the use of α-mangostin) for (the manufacture of a composition for) the treatment of muscular disorders and for the improvement of muscle function.

### DETAILED DESCRIPTION OF THE INVENTION

The expression “treatment” hereby also encompasses co-treatment, control, prevention and improvement, as well as maintenance of a healthy state.

The expression “prevention of a disorder” encompasses the reduction of the risk of coming down with said disorder and the reduction of the likelihood of the incidence of said disorder.

The expression “disorder” encompasses also diseases, as well as the individual subjective opinion that the current state needs to be improved.

We now found that compounds of the general formula I

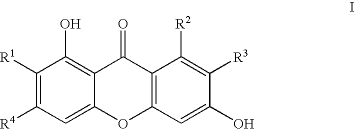

wherein R¹ and R² are independently from each other C₁₋₆-alk(en)yl and R³ and R⁴ are independently from each other hydroxy or C₁₋₆-alkyloxy;
may be effective in the prevention, control, and/or treatment of muscular disorders and the improvement of muscle function and other fields of use such as the treatment/prevention of eating disorders such as bulimia and anorexia nervosa, the acceleration of wound healing, as well as the treatment/prevention of bone disorders such as osteoporosis and osteopenia. The compounds of the general formula I, as well as compositions comprising one or more of them may be used for enhancing physical performance, for enhancing endurance capacity, for increasing muscle mass, for preventing muscle loss, for enhancing muscle recovery, for reducing muscle fatigue, for improving energy balance, for improving bone health and function and/or for improving wound healing.

The compounds of the general formula I are especially effective in the prevention, control, and/or treatment of muscular disorders and the improvement of muscle function. They are also especially suitable for enhancing physical performance, for enhancing endurance capacity, for increasing muscle mass, for preventing muscle loss, for enhancing muscle recovery, for reducing muscle fatigue, as well as for improving energy balance. The most preferred compound of formula I in this respect is α-mangostin.

The term “improvement of muscle function” encompasses the enhancement of the physical performance especially the enhancement of the physical endurance and the fatigue resistance.

Skeletal muscle fibers are generally classified as type I (oxidative/slow) or type II (glycolytic/fast) fibers. They display marked differences in respect to concentration, metabolism, and susceptibility to fatigue. Type I fibers are mitochondria-rich and mainly use oxidative metabolism for energy production, which provides a stable and long-lasting supply of ATP, and thus are fatigue-resistant. Type II fibers comprise three sub-types: IIa, IIx, and IIb. Type IIb fibers have the lowest levels of mitochondrial content and oxidative enzymes, rely on glycolytic metabolism as major energy source, and are susceptible to fatigue, while the oxidative and contraction functions of type IIa and IIx between type I and IIb. Adult skeletal muscle shows plasticity and can undergo conversion between different fiber types in response to exercise training or modulation of motoneuron activity (PLOS Biology 2004, 2(10), e294).

Determination of the muscle fiber composition in athletes revealed that elite endurance athletes have relatively more type I fibers than type II fibers in the trained musculature. Marathoners also tend to have more type I fibers. It was suggested that type I fiber might be a factor governing physical endurance capacity. In the contrary ageing and physical inactivity are conditions associated with a decrease in type I fibers, oxidative capacity and insulin sensitivity. It appears that the muscle oxidative capacity is a crucial factor for determining endurance and fatigue resistance. There seem to be an adaptive metabolic response of skeletal muscle to endurance exercise by controlling the number of oxidative muscle fibers (type I fibers).

The conversion of skeletal muscle fiber type IIb to type IIa and type I is regulated by different signaling pathways. For example the Ras/mitogen-activated protein kinase (MAPK), calcineurin, calcium/calmodulin-dependent protein kinase IV, and the peroxisome proliferatory coactivator 1 (PGC-1).

The compounds mentioned above may modulate these pathways and such may have an influence on the skeletal muscle fibers. Such an influence can be measured in intervention studies, e.g. in mice, to identify an effect on exercise performance, e.g. running distance. The effect on the skeletal muscle fibers can further be detected by histological (e.g. to determine the muscle fiber type) or molecular biology techniques (e.g. gene or protein expression).

Endurance capacity can be measured as the time to exhaustion when exercising at a constant workload or speed. The constant workload allows comparison of metabolic and other measurements in an experimental and a control condition. Performance trials have been developed for the treadmill. For example to quantify the enhancement of the physical endurance and the fatigue resistance the maximal running distance on a treadmill can be measured.

Endurance performance or endurance capacity tests may be divided into 3 categories: Constant work tests, usually referred to as time trials; Constant duration tests, these are similar to time trials but instead of completing a pre-set amount of work, as much work as possible is performed in a given time period; Constant power or constant load tests, these tests measure endurance capacity.

To measure increase in muscle mass body composition can be measured indirectly. Muscle and fat mass may be measured directly by scanning techniques (computerised tomography, nuclear magnetic resonance) or indirectly by anthropometry measurements such circumferences and skinfolds.

Such “diseases” connected to muscle disorders are muscle wasting and associated disorders such as sarcopenia, muscular damage, muscular dystrophies and muscular fatigue. The term “treatment of muscle disorders” also encompasses the maintenance of muscle performance and/or strength and muscle function. Moreover, such compounds can improve endurance, as well as muscle:fat ratio in individuals, who wish to do so, including in healthy individuals. In the most preferred embodiments of the present invention the compounds of the general formula I, especially α-mangostin, is used in these fields.

Muscle wasting is characterized by a progressive loss of muscle mass, weakening and degeneration of muscles especially the skeletal or voluntary muscles and the cardiac muscles. The processes by which atrophy and hypertrophy occur are conserved across mammalian species. Multiple studies have demonstrated that the same basic molecular, cellular, and physiological processes occur during atrophy in both rodents and humans.

Muscle wasting is due to a variety of causes and is associated with various pathologies, diseases and illnesses. These include but are not limited to muscular dystrophies caused by genetic disorders such as Duchenne's muscular dystrophy, progressive muscular dystrophy, Becker's type muscular dystrophy, Dejerine-Landouzy muscular dystrophy, Erb's muscular dystrophy, spinal muscular atrophy, and infantile neuroaxonal muscular dystrophy.

Muscles wasting can also be caused by a variety of chronic diseases and the ageing process. As the body ages, an increasing proportion of skeletal muscle is replaced by fibrous tissue. Therefore, normal aging in humans is associated with progressive decrease in skeletal muscle mass and strength, a condition called sarcopenia, which contributes to frailty and falls.

Moreover, age related disorders such as hypertension, glucose intolerance and diabetes, obesity, dyslipidemia, atherosclerotic and cardiovascular disease are also associated with loss of muscle mass.

In addition other conditions such as cancer, autoimmune diseases, infections disease, HIV infection, AIDS, malnutrition, renal diseases, osteomalacia, chronic lower back pain, peripheral nerve damage, spinal cord damage, chemical damage, central nervous system (CNS) damage are linked to or can cause muscle wasting. Finally, conditions resulting in muscle wasting may arise from disuse conditions such as long term immobilization due to illness or disability such as confinement in a wheelchair, prolonged bed rest, bone fracture or trauma. It is estimated that bed-rest after surgery causes loss of skeletal muscle mass of approximately 10% per week.

Untreated muscle wasting disorders can have serious health consequences. The changes that occur during muscle wasting can lead to a weakened physical state resulting in poor performance of the body and detrimental health effects.

Thus, muscle atrophy can seriously limit the rehabilitation of patients after immobilizations. Muscle wasting due to chronic diseases can lead to premature loss of mobility and increase the risk of disease-related morbidity. Muscle wasting due to disuse is an especially serious problem in elderly, who may already suffer from age-related deficits in muscle function and mass, leading to permanent disability and premature death as well as increased bone fracture rate. Despite the clinical importance of the condition few treatments exist to prevent or reverse the condition.

The compounds described herein (especially α-mangostin) may be useful for the prevention and treatment of muscle wasting leading to muscle loss and atrophy and the associated muscle disorders in mammals, in particular humans.

The present invention is directed to the use of the compounds of the general formula I as defined above (especially the use of α-mangostin) for
increasing fitness, physical endurance and physical performance; i.e. humans or animals to which the compounds are administered may be able to perform physical activities for a longer time than humans or animals to which the compounds were not administered;
improving skeletal muscle endurance and resistance to fatigue, the compounds are muscle remodelling agents, increasing the proportion of type I oxidative muscle fibres and stimulating mitochondrial biogenesis thus increasing muscle oxidative capacity which is a key factor for muscle endurance and muscle fatigue resistance;
preventing muscle mass loss; inhibiting muscle catabolism and increasing muscle anabolism, enhancing muscle recovery, reducing protein loss;
preventing sarcopenia; preventing frailty or age-related function decline due to aging, maintaining muscle strength and function in the elderly mammals;
improving muscle dystrophy, improve muscle disorders caused by a defect in one or more genes that control muscle function, decrease wasting of skeletal muscle;
increasing muscle metabolism to boost energy mobilization;
improving skeletal muscle mass by stimulating anabolic pathways, inhibiting catabolic pathways and accelerating muscle regeneration when damaged;
promoting fat burning; act as a regulator of fat burning, increasing energy expenditure by fatty acid oxidation, increasing fat metabolism, promoting fat oxidation, decreasing body fat and increasing muscle mass;
helping to achieve a good silhouette (body shaping), decreasing body fat and increasing lean muscle mass;
increasing thermogenesis; increasing the metabolism of a human or animal to burn more energy;
improving the energy balance, i.e. improving the energy balance of the whole body; whereby the energy balance is the balance between energy taken in, generally by food and beverage, and energy expended;
improving wound healing in conditions, where this is desirable;
promote skin health, especially accelerate/improve wound healing;
improve bone health and prevent bone-related disorders, such as osteoporosis and osteopenia.

Therefore, the present invention is directed to the use of compounds of the general formula I

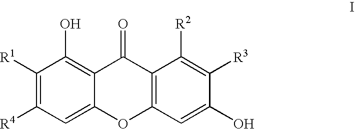

wherein R¹ and R² are independently from each other C₁₋₆-alk(en)yl and R³ and R⁴ are independently from each other hydroxy or C₁₋₆-alkyloxy (preferably R³ and R⁴ are independently from each other hydroxy or methoxy);
for the manufacture of compositions or medicaments, especially for the treatment of muscular disorders, for improving muscle function and endurance, for the treatment of eating disorders such as bulimia and anorexia nervosa, for accelerating wound healing, and/or for treating bone disorders such as osteoporosis and osteopenia.

The composition manufactured by use of (one or more) compounds of the general formula I is used especially for enhancing physical performance, for enhancing endurance capacity, for increasing muscle mass, for preventing muscle loss, for enhancing muscle recovery, for reducing muscle fatigue, for improving energy balance, for improving bone health and function and/or for improving wound healing, as well as for the maintenance of muscle performance and/or muscle strength and/or muscle mass and/or muscle function, and/or for improving the body shape and/or for improving the muscle:fat ratio in mammals including humans.

The composition is preferably a dietary, body care or pharmaceutical composition as defined below. The dietary composition is preferably in form of food such as dairy products (e.g. yoghurts), in form of fortified food such as cereal bars and bakery items such as cakes and cookies, in form of dietary supplements such as tablets, pills, granules, dragées, capsules, and effervescent formulations, in form of non-alcoholic drinks such as instant drinks, soft drinks, sport drinks or sport beverages in general, fruit juices, vegetable juices, smoothies, lemonades, teas, functional water, near-water drinks and milk based drinks, in form of liquid food such as soups and dairy products (muesli drinks), as defined below.

The present invention is further directed to the use of compounds of the general formula I

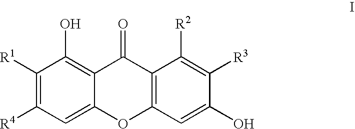

wherein R¹ and R² are independently from each other C₁₋₆-alk(en)yl and R³ and R⁴ are independently from each other hydroxy or C₁₋₆-alkyloxy (preferably R³ and R⁴ are independently from each other hydroxy or methoxy);
for the treatment of muscular disorders, for improving muscle function and endurance, for enhancing physical performance, for enhancing endurance capacity, for increasing muscle mass, for preventing muscle loss, for enhancing muscle recovery, for reducing muscle fatigue, for improving energy balance, for the treatment of eating disorders such as bulimia and anorexia nervosa, for accelerating/improving wound healing, and/or for treating bone disorders such as osteoporosis and osteopenia, as well as for improving bone health and function in general.

Preferably the present invention is directed to the use of a compound of the formula I, wherein R¹ and R² are both isoprenyl and wherein R³ and R⁴ are both methoxy (this compound is called “α-mangostin”):

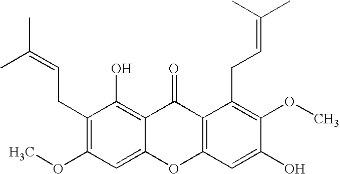

for the treatment of muscular disorders, for improving muscle function and endurance, for enhancing physical performance, for enhancing endurance capacity, for increasing muscle mass, for preventing muscle loss, for enhancing muscle recovery, for reducing muscle fatigue, for improving energy balance, for the treatment of eating disorders such as bulimia and anorexia nervosa, for accelerating/improving wound healing, and/or for treating bone disorders such as osteoporosis and osteopenia, as well as for improving bone health and function in general.

In preferred embodiments of the invention the compound of the formula I with the definitions of R¹ to R⁴ and the preferences as given above is the only compound of Garcinia mangostana L./mangosteen present in the compositions/medicaments.

The term “compound of the formula I” also encompasses any material or extract of a plant or any material or extract of parts of a plant or any material or extract of fruits of a plant containing such a compound of the formula I, preferably in an amount of at least 1, 5, 10, 15, 20, 25, 30 weight-%, more preferably in an amount of at least 50, 60, 70, 80 weight-%, even more preferably in an amount of at least 90 weight-%, most preferably in an amount of at least 95 weight-%, based on the total weight of the plant material or extract. The terms “material of a plant” and “plant material” used in the context of the present invention mean any part of a plant including its flowers and fruits.

α-Mangostin (1,3,6-trihydroxy-7-methoxy-2,8-bis(3-methyl-2-butenyl)-9H-xanthen-9-one) can be isolated from plants like Garcinia mangostana (see Journal of American Chemical Society 1958, 80, 1691 ff.; Phytochemistry 1996, 43(5), 1099-1102), but not limited to them.

Therefore, any material or extract of these plants or any other plant material or extract containing α-mangostin, preferably in an amount of at least 1, 5, 10, 15, 20, 25, 30 weight-%, more preferably in an amount of at least 50, 60, 70, 80 weight-%, even more preferably in an amount of at least 90 weight-%, most preferably in an amount of at least 95 weight-%, based on the total weight of the plant material or extract is also encompassed by this expression. “α-Mangostin” means both “natural” (isolated) and “synthetic” (manufactured) α-mangostin.

Beside the (pure) compound α-mangostin preferred are plant materials and plant extracts, especially those containing at least 10, 15, 20, 25, 30 weight-%, preferably at least 50, 60, 70, 80 weight-%, more preferably at least 90 weight-%, most preferably in an amount of at least 95 weight-%, of these compounds, based on the total weight of the plant material/extract.

In preferred embodiments of the present invention the compound of the general formula I (especially α-mangostin) is used as physical performance enhancer, as endurance increaser, and as muscle loss decreasing agent.

The present invention is also directed to a composition (especially a dietary, personal care or pharmaceutical composition) containing at least a compound of the formula I,

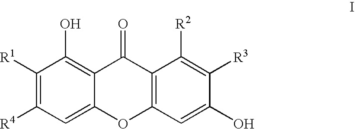

wherein R¹ and R² are independently from each other C₁₋₆-alk(en)yl (preferably both are isoprenyl) and R³ and R⁴ are independently from each other C₁₋₆-alkyloxy (preferably R³ and R⁴ are independently from each other hydroxy or methoxy, more preferably both are methoxy), as well as to the use of such compositions for the treatment of muscular disorders, for improving muscle function and endurance, for enhancing physical performance, for enhancing endurance capacity, for increasing muscle mass, for preventing muscle loss, for enhancing muscle recovery, for reducing muscle fatigue, for improving energy balance, for the treatment of eating disorders such as bulimia and anorexia nervosa, for accelerating/improving wound healing, and/or for treating bone disorders such as osteoporosis and osteopenia, as well as for improving bone health and function in general. Encompassed by the present invention is also the use of such compositions comprising a compound of the general formula I as defined above (especially compositions comprising α-mangostin) for the maintenance of muscle performance and/or muscle strength and/or muscle mass and/or muscle function, and/or for improving the body shape and/or for improving the muscle:fat ratio in mammals including humans.

If the composition is a dietary composition it is preferably in form of dairy products, in form of fortified cereal bars or fortified bakery items, in form of tablets, pills, granules, dragées, capsules, or effervescent formulations, in form of instant drinks, soft drinks, sport drinks, sport beverages in general, fruit juices, vegetable juices, smoothies, lemonades, teas, functional water, near-water drinks or milk based drinks, or in form of soups.

The present invention is further directed to a composition (especially a dietary, personal care or pharmaceutical composition) as defined above comprising a compound of the general formula I as defined above (especially comprising α-mangostin) for all the uses as listed above.

In the field of muscular disorders (encompassing also improving muscle function and endurance, enhancing physical performance, enhancing endurance capacity, increasing muscle mass, preventing muscle loss, enhancing muscle recovery, reducing muscle fatigue, improving energy balance), in the field of eating disorders and in the field of bone disorders the compositions, especially the dietary and pharmaceutical compositions, are administered preferably orally to the subjects in need thereof and as defined below (mammals including humans). In the field of wound healing the compositions, especially the body care compositions, are preferably administered topically to the subjects in need thereof and as defined below (mammals including humans).

In preferred embodiments of the compositions of the present invention the compound of the formula I with the definitions of R¹ to R⁴ and the preferences as given above is the only compound of Garcinia mangostana L./mangosteen present in the compositions.

The term “dietary compositions” comprises any type of (fortified) food, (fortified) (animal) feed, feed premixes, food premixes, sport food, and beverages including also clinical nutrition, and also dietary supplements as well as the corresponding additives: food additives, beverage additives, feed additives. Also encompassed is functional food/feed i.e. a food/feed that has been enhanced with vitamins, other micronutrients or pharmaceuticals to provide further specific health benefits, as well as a nutraceutical, i.e. a pill or other pharmaceutical product that has nutritional value.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellyfying agents, gel forming agents, antioxidants and antimicrobials.

The present invention is also directed to a pharmaceutical composition containing at least one compound of the formula I with the definitions of R¹ to R⁴ and the preferences as given above and a conventional pharmaceutical carrier.

Beside a pharmaceutically acceptable carrier and at least one compound of the formula I with the definitions of R¹ to R⁴ and the preferences as given above, the pharmaceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The carrier material can be organic or inorganic inert carrier material suitable for oral/paren-teral/injectable administration.

The dietary and pharmaceutical compositions according to the present invention may be in any galenic form that is suitable for administering to the animal body including the human body, especially in any form that is conventional for oral administration, e.g. in solid form such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be filled into hard or soft shell capsules. Examples for other application forms are forms for transdermal, parenteral or injectable administration. The dietary and pharmaceutical compositions may be in the form of controlled (delayed) release formulations.

Examples of food/feed additives are any composition/formulation added to food/feed during its manufacture or its preparation for consumption.

Examples for fortified food are cereal bars, bakery items such as cakes and cookies.

Beverages encompass non-alcoholic and alcoholic drinks as well as liquid preparations to be added to drinking water and liquid food. Non-alcoholic drinks are e.g. instant drinks, soft drinks, sport drinks or sport beverages in general, fruit juices such as e.g. orange juice, apple juice and grapefruit juice; vegetable juices such as tomato juice; smoothies, lemonades, functional water, near-water drinks (i.e. water based drinks with a low calorie content), teas and milk based drinks. Liquid food are e.g. soups and dairy products such as muesli drinks.

Sports drinks are beverages designed to help subjects re-hydrate and are intended to replenish electrolytes, sugar, water, and other nutrients, which can be depleted after strenuous exercise. More effective than just water, sports drinks may also be used to treat dehydration due to illness if oral re-hydration therapy is unavailable.

The sports drinks can be hypotonic, hypertonic or isotonic. Sports drinks can be available in liquid form, as concentrates or as powder (to be dissolved in a liquid, as for example water). The sports food can be in the form of bars, tablets and gels.

“Functional waters” are near-water drinks with health benefits.

A major category of functional water-based beverages is the nutraceutical market where also various types of fruit or herbal concentrates are added to water for nutrient value.

The compounds of the formula I with the definitions of R¹ to R⁴ and the preferences as given above as well as (mixtures of) plant materials and plant extracts containing them, preferably in an amount of at least 1, 5, 10, 15, 20, 25, 30 weight-%, more preferably in an amount of at least 50, 60, 70, 80 weight-%, even more preferably in an amount of at least 90 weight-%, most preferably in an amount of at least 95 weight-%, based on the total weight of the plant material or extract, and dietary/pharmaceutical compositions containing them are thus suitable for the treatment of mammals including humans.

The present invention is also directed to personal care compositions containing at least one compound of the formula I with the definitions of R¹ to R⁴ and the preferences as given above and a conventional cosmetic carrier. Personal care compositions encompass skin care preparations, preparations containing scents and/or fragrances, preparation, hair-care preparations, dentrifices, deodorant and antiperspirant, decorative preparations, light protection preparations and functional preparations, as well as preparations promoting/for improved wound healing/skin regeneration.

The compounds of the formula I with the definitions of R¹ to R⁴ and the preferences as given above as well as (mixtures of) plant materials and plant extracts containing them, preferably in an amount of at least 1, 5, 10, 15, 20, 25, 30 weight-%, more preferably in an amount of at least 50, 60, 70, 80 weight-%, even more preferably in an amount of at least 90 weight-%, most preferably in an amount of at least 95 weight-%, based on the total weight of the plant material or extract, and body care compositions containing them are thus suitable for the topical treatment of mammals including humans.

The invention further relates to a method for the treatment of muscular disorders (preferred), for improving muscle function and endurance (preferred), for the treatment of eating disorders such as bulimia and anorexia nervosa, for accelerating wound healing, and/or for treating bone disorders such as osteoporosis and osteopenia in mammals including humans, said method comprising administering an effective dose of a compound of the formula I with the definitions of R¹ to R⁴ and the preferences as given above to mammals including humans which are in need thereof. The present invention is also preferably directed to methods of maintaining the healthy state and/or improving the baseline conditions concerning muscle function/muscle performance/muscle strength/muscle mass, said method comprising administering an effective dose of a compound of the formula I with the definitions of R¹ to R⁴ and the preferences as given above to mammals including humans which are in need thereof.

Mammals in the context of the present invention include humans. Preferred “mammals” are humans, farm animals such as horses, sheep, pigs/swines, cattle, cows and goats; and pets such as cats, dogs, horses, dromedaries, and elephants, especially dogs. Especially preferred mammals are humans. These preferences apply for all uses of the present invention.

In the context of this invention “treatment” also encompasses co-treatment as well as control and or prevention. In the context of this invention the term “disorder” also encompasses diseases. Furthermore, “treatment” also encompasses the use by healthy individuals, who seek for better fitness, body shape, or skin appearance.

For humans a suitable daily dosage of a compound of the formula I with the definitions of R¹ to R⁴ and the preferences as given above and preferably to be administered orally, for the purposes of the present invention may be within the range from 0.01 mg per kg body weight to 50 mg per kg body weight per day, i.e. 0.7 mg-3500 mg for a 70 kg person. More preferred is a daily dosage of 0.1 to 25 mg per kg body weight (i.e. 7 mg-1750 mg for a 70 kg person), and especially preferred is a daily dosage of 0.3 to 15 mg per kg body weight, i.e. 21 mg-1050 mg for a 70 kg person. The amount of a plant material or plant extract containing such compound of the formula I can be calculated accordingly.

In solid dosage unit preparations for humans, especially for oral administration to humans, the compound of the formula I with the definitions of R¹ to R⁴ and the preferences as given above is suitably present in an amount from 0.25 mg to 1000 mg, preferably from 2 mg to 200 mg per dosage unit.

In dietary compositions, especially in food and beverages for humans, the compound of the formula I with the definitions of R¹ to R⁴ and the preferences as given above may suitably be present in an amount of from 7 mg to 1750 mg, preferably, from 20 mg to 1000 mg per serving (serving size can be e.g. 500 mg for a lozenge, 50 g for bread or 250 mL for a beverage).

In food and drinks in a preferred embodiment of the invention the amount of the compound of the formula I with the definitions of R¹ to R⁴ and the preferences as given above may be 20 mg to 1000 mg per serving.

For dogs a suitable daily dosage of a compound of the formula I with the definitions of R¹ to R⁴ and the preferences as given above for the purposes of the present invention, preferably to be administered orally, may be within the range from 0.04 mg per kg body weight to 500 mg per kg body weight per day. More preferred is a daily dosage of 0.4 mg to 100 mg per kg body weight, and especially preferred is a daily dosage of 1 mg to 50 mg per kg body weight.

The present invention is also directed to the use of compounds of the general formula I

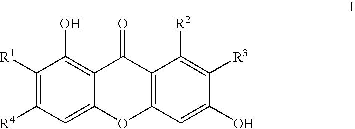

wherein R¹ and R² are independently from each other C₁₋₆-alk(en)yl (preferably both are isoprenyl) and R³ and R⁴ are independently from each other hydroxy or C₁₋₆-alkyloxy (preferably R³ and R⁴ are independently from each other hydroxy or methoxy, more preferably both are methoxy) as medicament, as well as to the compounds themselves.

The invention is illustrated further by the following examples.

### EXAMPLES

### Example 1

### Soft Gelatin Capsule

Soft gelatin capsules are prepared by conventional procedures providing a dose of compound of the formula I of 200 mg. Other ingredients: glycerol, water, gelatine, vegetable oil. A suitable daily dose is 1 to 5 capsules to increase the performance without having to change the exercise habits during the supplementation period. The supplementation should start a few weeks before the full effect can be achieved.

### Example 2

### Hard Gelatin Capsule

Hard gelatin capsules are prepared by conventional procedures providing a dose of compound of the formula I of 100 mg. A suitable daily dose is 1 to 5 capsules.

### Other Ingredients:

Fillers: lactose or cellulose or cellulose derivatives q.s.
Lubricant: magnesium stearate if necessary (0.5%)

### Example 3

### Tablet

Tablets are prepared by conventional procedures providing as active ingredient 50 mg of compound of the formula I per tablet, and as excipients microcrystalline cellulose, silicone dioxide (SiO₂), magnesium stearate, crospovidone NF (which is a disintegration agent) ad 500 mg. The consumption of 2 to 5 tablets a day over several months may result in a reduction of the body fat ratio without having to change dietary and exercise habits.

### Example 4

### Orange Juice Drink Coloured with 30 mg β-Carotene 10% CWS

| Ingredients | [g] |
|---|---|
| Sugar syrup 64° Brix | 156.2 |
| Sodium benzoate | 0.2 |
| Ascorbic acid, fine powder | 0.2 |
| Citric acid 50% w/w | 5.0 |
| Pectin solution 2% w/w | 10.0 |
| compound of the formula I | 0.5 |
| Juice compound* | 30.0 |
| Water to | 250.0 |

### Preparation

Dissolve sodium benzoate in water whilst stirring;
Continue stirring and add sugar syrup, ascorbic acid, citric acid, pectin solution, juice compound, one after the other. Do not use a high speed mixer;
Dilute the bottling syrup with (carbonated) water to one liter of beverage.

| *Ingredients Juice compound | [g] |
|---|---|
| Orange juice concentrate 65° Brix | 483.3 |
| Lemon Juice Concentrate 45° Brix | 173.3 |
| Oily orange flavour | 5.0 |
| β-Carotene 10% CWS as 10% stocksolution | 10.0 |
| Deionized water | 328.4 |

### Preparation of Juice Compound

Add the deionized water to the juice concentrates, stir gently and allow the juice concentrates to hydrate.

Add the oily flavour and β-Carotene 10% CWS stocksolution and pre-emulsify in a rotor-stator-homogenizer.

Homogenize in a high-pressure homogenizer at 200 bar.

### Addition of β-Carotene 10% CWS

β-Carotene 10% CWS should be added to the juice compound as a 1-10% stocksolution in deionized water.

The orange juice drink contains 3 ppm β-carotene.

### Example 5

### Influence of α-Mangostin on the Running Performances on the Body Composition and on the Gastrocnemius-Plantaris-Soleus Muscle Group of Mice

20 male C57B1/6J mice were obtained from Jackson Laboratory (Bar Harbor, Me., USA) at the age of 5 weeks. All mice were administered a high-fat diet (Kliba#2154, Provimi Kliba AG, Kaiseraugst, Switzerland) for 8 weeks to induce obesity. Thereafter, the mice were randomly assigned into two groups:

Group 1: Control (high-fat diet)
Group 2: alpha-Mangostin (high-fat diet supplemented with 0.03% w/w alpha-Mangostin (Indofine Chemical Company))

The duration of the supplementation was 9 weeks. During this period all mice received food and water ad libitum. At the end of the supplementation period, maximal running performance on a rodent treadmill (Technical & Scientific Equipment GmbH, Bad Homburg, Germany) was determined. Body composition was measured by quantitative magnetic resonance (Minispec MQ10, Bruker Optics GmbH, Faellanden, Switzerland) in conscious animals. At the end of the study, the animals were killed, blood was taken and the gastrocnemius-plantaris-soleus muscle group was excised and weighted.

Supplementation of mice with alpha-Mangostin decreased body weight and the percentage of body fat mass while it increased the percentage of lean body mass compared to mice in the control group (Table 1). Furthermore, the weight of the gastrocnemius-plantaris-soleus muscle group relative to body weight was increased by alpha-Mangostin consumption and maximal running distance increased by 9% (Table 1).

TABLE 1
Body weight, body fat mass, lean body mass, gastrocnemius-plantaris-
soleus weight and maximal running distance of control mice and
mice supplemented with alpha-Mangostin.

| | Control | alpha-Mangostin |
|---|---|---|
| Body weight (g) | 33.1 | 27.3 |
| Body fat mass (% of body weight) | 22.7 | 7.2 |
| Lean body mass (% of body weight) | 69.7 | 80.6 |
| Gastrocnemius-plantaris-soleus weight (mg/g | 6.06 | 6.70 |
| body weight) | | |
| Maximal running distance (m) | 2081 | 2268 |

Increased maximal running distance in a treadmill test demonstrates enhanced endurance which is an indicator of improved oxidative capacity in skeletal muscle caused by the consumption of alpha-mangostin. Anatomically, this is reflected by an increased proportion of type I and type IIa muscle fibers. Furthermore, consumption of alpha-mangostin reduced body fat mass and increased lean body mass compared to control animals, thereby ameliorating the deleterious effects of the high-fat diet. This effect can be caused by increased fat oxidation in skeletal muscle due to a greater proportion of oxidative type I and type IIa muscle fibers.

Therefore, supplementation with alpha-mangostin enhances muscle function and endurance and improves the body shape as well as the muscle:fat ratio in mammals.

1. Use of a compound of the general formula I 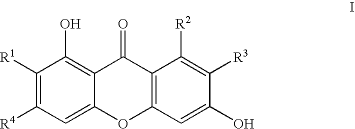
wherein R¹ and R² are independently from each other C₁₋₆alk(en)yl and R³ and R⁴ are independently from each other hydroxy or C₁₋₆-alkyloxy;
for the manufacture of a composition for the treatment of muscular disorders, for improving muscle function and endurance, for the treatment of eating disorders such as bulimia and anorexia nervosa, for accelerating wound healing, and/or for treating bone disorders such as osteoporosis and osteopenia.

2. The use according to claim 1, wherein the composition is used for enhancing physical performance, for enhancing endurance capacity, for increasing muscle mass, for preventing muscle loss, for enhancing muscle recovery, for reducing muscle fatigue, for improving energy balance, for improving bone health and function and/or for improving wound healing.

3. The use according to claim 1, wherein the composition is used for the maintenance of muscle performance and/or muscle strength and/or muscle mass and/or muscle function, and/or for improving the body shape and/or for improving the muscle:fat ratio in mammals including humans.

4. The use according to claim 1, wherein the composition is a dietary, body care or pharmaceutical composition.

5. The use according to claim 1, characterized in that the compound of the general formula I is one, wherein R¹=R²=iso-prenyl and R³=R⁴=methoxy.

6. The use according to claim 4 wherein the dietary composition is in form of food such as dairy products (e.g. yoghurts), in form of fortified food such as cereal bars and bakery items such as cakes and cookies, in form of dietary supplements such as tablets, pills, granules, dragées, capsules, and effervescent formulations, in form of non-alcoholic drinks such as instant drinks, soft drinks, sport drinks or sport beverages in general, fruit juices, vegetable juices, smoothies, lemonades, teas, functional water, near-water drinks and milk based drinks, in form of liquid food such as soups and dairy products (muesli drinks).

7. Composition, especially dietary, body care or pharmaceutical composition, comprising a compound of general formula I, 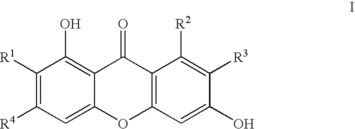
wherein R¹ and R² are independently from each other C₁₋₆-alk(en)yl and R³ and R⁴ are independently from each other hydroxy or C₁₋₄-alkyloxy.

8. Compound of general formula I, 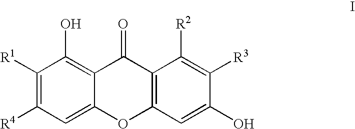
wherein R¹ and R² are independently from each other C₁₋₄-alk(en)yl and R³ and R⁴ are independently from each other hydroxy or C₁₋₆-alkyloxy.

9. A method for the treatment and/or prevention of muscular disorders, for the improvement of muscle function and endurance, for the treatment and/or prevention of eating disorders such as bulimia and anorexia nervosa, for accelerating wound healing, and/or for the treatment and/or prevention of bone disorders such as osteoporosis and osteopenia, in mammals including humans, said method comprising administering an effective dose of a compound of the general formula I as defined in claim 1, preferably of a compound as defined in the compound of the general formula I is one, wherein R¹=R²=iso-prenyl and R³=R⁴=methoxy, to mammals including humans which are in need thereof.

10. The method according to claim 9, wherein the mammal is a human, a pet animal or a farm animal.

11. The method according to claim 9, wherein the mammal is a human.

12. Use of a compound of the general formula I 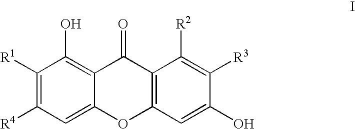
wherein R¹ and R² are independently from each other C₁₋₆-alk(en)yl and R³ and R⁴ are independently from each other hydroxy or C₁₋₆-alkyloxy;
for the manufacture of a composition for the treatment of muscular disorders and/or for improving muscle function and endurance in mammals including humans.

13. Use of a composition comprising a compound of the general formula I 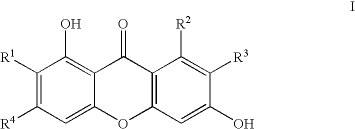
wherein R¹ and R² are independently from each other C₁₋₆-alk(en)yl and R³ and R⁴ are independently from each other hydroxy or C₁₋₆-alkyloxy;
for the treatment of muscular disorders and/or for improving muscle function and endurance in mammals including humans.

14. The use according to claim 12, wherein the (composition comprising the) compound is used for enhancing physical performance, for enhancing endurance capacity, for increasing muscle mass, for preventing muscle loss, for enhancing muscle recovery, for reducing muscle fatigue and/or for improving energy balance.

15. The use according to claim 14, wherein the (composition comprising the) compound is used for the maintenance of muscle performance and/or muscle strength and/or muscle mass and/or muscle function, and/or for improving the body shape and/or for improving the muscle:fat ratio in mammals including humans.

16. The use according to claim 13, wherein the composition is a dietary, body care or pharmaceutical composition.

17. The use according to claim 12, characterized in that the compound of the general formula I is one, wherein R¹=R²=iso-prenyl and R³=R⁴=methoxy.

18. The use according to claim 16 wherein the dietary composition is in form of dairy products, in form of fortified cereal bars or fortified bakery items, in form of tablets, pills, granules, dragées, capsules, or effervescent formulations, in form of instant drinks, soft drinks, sport drinks, sport beverages in general, fruit juices, vegetable juices, smoothies, lemonades, teas, functional water, near-water drinks or milk based drinks, or in form of soups.

19. Composition, especially dietary or pharmaceutical composition, comprising a compound of general formula I, 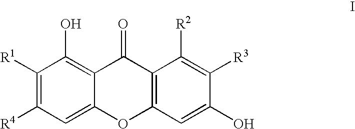
wherein R¹ and R² are independently from each other C₁₋₆-alk(en)yl and R³ and R⁴ are independently from each other hydroxy or C₁₋₄-alkyloxy, for the treatment of muscular disorders and/or for improving muscle function and endurance in mammals including humans.

20. Composition, especially dietary or pharmaceutical composition, comprising a compound of general formula I, 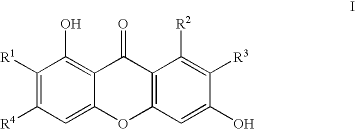
wherein R¹ and R² are independently from each other C₁₋₄-alk(en)yl and R³ and R⁴ are independently from each other hydroxy or C₁₋₄-alkyloxy, for the treatment of eating disorders such as bulimia and anorexia nervosa in mammals including humans.

21. Composition, especially body care composition, comprising a compound of general formula I, 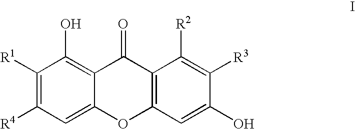
wherein R¹ and R² are independently from each other C₁₋₆-alk(en)yl and R³ and R⁴ are independently from each other hydroxy or C₁₋₆-alkyloxy, for accelerating and/or improving wound healing in mammals including humans.

22. Composition, especially dietary or pharmaceutical composition, comprising a compound of general formula I, 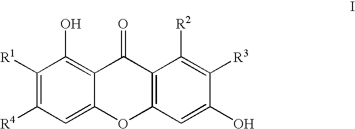
wherein R¹ and R² are independently from each other C₁₋₆(en)yl and R³ and R⁴ are independently from each other hydroxy or C₁₋₆-alkyloxy, for treating bone disorders such as osteoporosis and osteopenia and/or for improving bone health and function in mammals including humans.

23. Compound of general formula I, 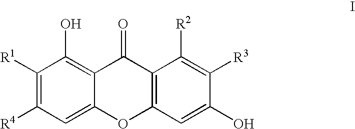
wherein R¹ and R² are independently from each other C₁₋₆alk(en)yl and R³ and R⁴ are independently from each other hydroxy or C₁₋₄-alkyloxy, for the treatment of muscular disorders and/or for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans.

24. Compound of general formula I, 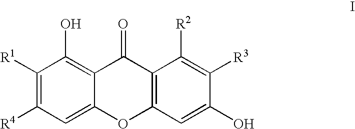
wherein R¹ and R² are independently from each other C₁₋₄-alk(en)yl and R³ and R⁴ are independently from each other hydroxy or C₁₋₆alkyloxy, for the treatment of eating disorders such as bulimia and anorexia nervosa in mammals including humans.

25. Compound of general formula I, 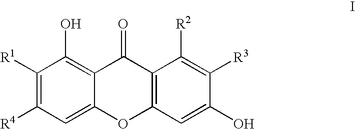
wherein R¹ and R² are independently from each other C₁₋₄-alk(en)yl and R³ and R⁴ are independently from each other hydroxy or C₁₋₄-alkyloxy, for accelerating and/or improving wound healing in mammals including humans.

26. Compound of general formula I, 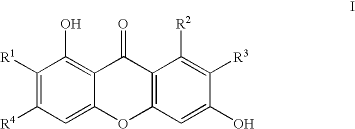
wherein R¹ and R² are independently from each other C₁₋₄-alk(en)yl and R³ and R⁴ are independently from each other hydroxy or C₁₋₆alkyloxy, for treating bone disorders such as osteoporosis and osteopenia and/or for improving bone health and function in mammals including humans.

27. A method for the treatment and/or prevention of muscular disorders and/or for the improvement of muscle function and endurance in mammals including humans, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans, said method comprising administering an effective dose of a compound of the general formula I as defined in claim 1, preferably of a compound as defined in that the compound of the general formula I is one, wherein R¹=R²=iso-prenyl and R³=R⁴=methoxy, to mammals including humans which are in need thereof.

28. The method according to claim 27, wherein the mammal is a human, a pet animal or a farm animal.

29. The method according to claim 27, wherein the mammal is a human.

30. Use of a compound of the general formula I 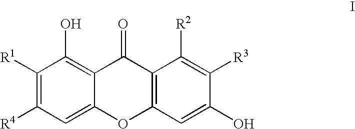
wherein R¹ and R² are independently from each other C₁₋₆-alk(en)yl and R³ and R⁴ are independently from each other hydroxy or C₁₋₆alkyloxy (preferably R³ and R⁴ are independently from each other hydroxy or methoxy);
for the treatment of muscular disorders, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans.

31. Use of a compound of the general formula I 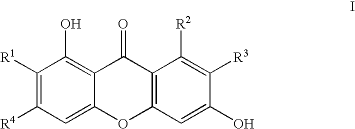
wherein R¹ and R² are independently from each other C₁₋₆(en)yl and R³ and R⁴ are independently from each other hydroxy or C₁₋₆-alkyloxy; for the treatment of eating disorders such as bulimia and anorexia nervosa.

32. Use of a compound of the general formula I 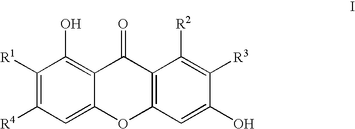
wherein R¹ and R² are independently from each other C₁₋₆-alk(en)yl and R³ and R⁴ are independently from each other hydroxy or C₁₋₆-alkyloxy; for accelerating and/or improving wound healing in mammals including humans.

33. Use of a compound of the general formula I 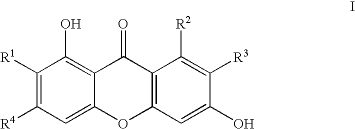
wherein R¹ and R² are independently from each other C₁₋₆-alk(en)yl and R³ and R⁴ are independently from each other hydroxy or C₁₋₆-alkyloxy; for treating bone disorders such as osteoporosis and osteopenia in mammals including humans.

34. The use according to claim 33, wherein the compound is used for improving bone health and function.

35. The use according to claim 30, characterized in that the compound of the general formula I is one, wherein R^{1=R}²=iso-prenyl and R³=R⁴=methoxy.

36. A method for the treatment and/or prevention of eating disorders such as bulimia and anorexia nervosa in mammals including humans, said method comprising administering an effective dose of a compound of the general formula I as defined in claim 1, preferably of a compound characterized in that the compound of the general formula I is one, wherein R¹=R² iso-prenyl and R³=R⁴=methoxy, to mammals including humans which are in need thereof.

37. A method for accelerating/improving wound healing in mammals including humans, said method comprising administering an effective dose of a compound of the general formula I as defined in claim 1, preferably of a compound characterized in that the compound of the general formula I is one, wherein R¹=R²=iso-prenyl and R³=R⁴=methoxy, to mammals including humans which are in need thereof.

38. A method for the treatment and/or prevention of bone disorders such as osteoporosis and osteopenia in mammals including humans and/or for improving bone health and function in mammals including humans, said method comprising administering an effective dose of a compound of the general formula I as defined in claim 1, preferably of a compound characterized in that the compound of the general formula I is one wherein R¹=R²=iso-prenyl and R³=R⁴=methoxy, to mammals including humans which are in need thereof.

39. The method according to claim 36, wherein the mammal is a human, a pet animal or a farm animal, especially wherein the mammal is a human.
